(19) United States
(12) Patent Application Publication
(10) Pub. No.: US 2004/0191274 A1
(43) Pub. Date: Sep. 30, 2004

(54) Topical composition

(76) Inventors: Michael L. Grayson, Kew (AU); Suzanne W. Kirsa, Blackburn South (AU); Alan D. Shilson, Viewbank (AU)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/403,403

(22) Filed: Mar. 31, 2003

(51) Int. Cl. A61K 9/00; A61K 31/205

(52) U.S. Cl. 424/400; 514/554

(57) ABSTRACT

A topical composition comprising at least one C₁ to C₄ alcohol, at least one anti-microbial and at least one emollient.

### FIELD OF THE INVENTION

The present invention relates to topical compositions, which may be used as an antiseptic and/or disinfectant.

### BACKGROUND OF THE INVENTION

Control of nosocomial infection and exposure to infectious disease is of paramount concern to doctors, nurses, and clinicians that work in hospitals, clinics and surgical and medical centers. One of the most effective methods for controlling infection is regimented hand disinfection before and after each patient contact and before invasive procedures.

Hand disinfection is generally accomplished using anti-microbial soaps with water. These soaps are usually formulated to include either povidone-iodine or chlorhexidine gluconate as the active anti-microbial agent. In addition, these formulated soaps may contain surfactants and possibly low levels of humectants such as glycerin.

Hand disinfection is also accomplished using hand wash replacements. These are used instead of the soap and water scrub. Hand wash replacements ideally achieve bacterial kill equal to or better than a traditional soap and water scrub and in a shorter period of time. Additionally, they maintain or improve the skin's natural barrier to microbial and chemical contamination while providing acceptable tactile properties. Examples of hand wash replacements include hydroalcoholic gels, which generally include high levels of either ethanol or isopropanol as the disinfecting agent and also include a thickener and/or surfactant and optionally include a humectant (e.g. glycerin).

### SUMMARY OF THE INVENTION

The present invention provides a topical composition comprising:

at least one C₁ to C₄ alcohol;

at least one anti-microbial; and

at least one emollient.

The C₁ to C₄ alcohols may include straight or branched chain alcohols. Preferably, the alcohols are selected from ethanol, n-propanol and isopropanol. Preferably, the alcohol is isopropanol.

The alcohol concentration is preferably about 60% v/v to about 90% v/v, more preferably about 65% v/v to about 75% v/v, most preferably about 70% v/v of the total concentration of the topical composition.

The anti-microbial may be selected from chlorhexidine and its salts. A preferred anti-microbial is chlorhexidine gluconate.

The anti-microbial is preferably present in an amount of about 3 g/L to about 10 g/L, more preferably about 4 g/L to about 7 g/L, most preferably about 5 g/L of the topical composition.

The emollient may be selected from general emollients, occlusive emollients and humectants.

Preferably, the emollient is a humectant. More preferably, the emollient is selected from humectants such as glycerol, propylene glycol, dipropylene glycol, polypropylene glycol, polyethylene glycol, sorbitol, pantothenol and gluconic acid salts. Most preferably, the emollient is polyethylene glycol.

The concentration of the emollient in the topical composition is preferably about 1.0% to about 2.0%, more preferably about 1.5% to about 1.8%, most preferably about 1.5%.

The present invention also provides a method of reducing and/or preventing the transmission of a microorganism by applying an effective amount of a topical composition as defined above to any part of the body excluding mucous membranes.

The topical composition as defined above may therefore be used to reduce and/or prevent the transmission of a microorganism.

The topical composition may also be used in the manufacture of a disinfectant and/or antiseptic to reduce and/or prevent the transmission of a microorganism.

The microorganism is preferably a multi-resistant organism such as, for example, methicillin-resistant staphylococcus aureus.

The topical composition may be applied before and after routine or specialized health-care delivery or preparatory to invasive procedures.

An effective amount of the topical composition is preferably about 1 ml to about 3 ml.

The present invention further provides a method of preparing a topical composition as defined above comprising the step of mixing at least one C₁ to C₄ alcohol, at least one anti-microbial, and at least one emollient, with stirring, until complete homogenization is achieved.

### DETAILED DESCRIPTION OF PREFERRED EMBODIMENT

The present invention provides a topical composition comprising at least one C₁ to C₄ alcohol, at least one anti-microbial, and at least one emollient.

Alcohols

Alcohol is an effective vehicle for a disinfectant. It provides a broad spectrum bactericidal effect and is fast drying.

The C₁ to C₄ alcohols in the topical composition of the present invention may include straight or branched chain alcohols. Suitable alkanols include ethanol, n-propanol and isopropanol.

While the topical composition of the present invention may comprise only one alcohol, a combination of alcohols may be used.

Isopropanol is a good disinfectant with a pleasant odor and, compared to other alcohols, it has a less cutaneous drying effect of the skin. Therefore, isopropanol is a preferred alcohol for the topical composition of the present invention.

The alcohol concentration of the topical composition should provide the greatest possible bacterial kill effect. Therefore, the alcohol concentration is preferably about 60% v/v to about 90% v/v, more preferably about 65% v/v to about 75% v/v, most preferably about 70% v/v of the total concentration of the topical composition.

Anti-Microbials

The term “anti-microbials” is used herein in its broadest sense and refers to any agent that can treat any infection caused by a microorganism and includes viral and bacterial infections. Examples of such infectious microorganisms may be found in a number of well known texts such as ‘Medical Microbiology’ (Greenwood, D., Slack, R., Peutherer, J., Churchill Livingstone Press, 2002); ‘Mims’ Pathogenesis of Infectious Disease’ (Mims, C., Nash, A., Stephen, J., Academic Press, 2000); “Fields” Virology. (Fields, B. N., Knipe, D. M., Howley, P. M., Lippincott Williams and Wilkins, 2001).

Chlorhexidine and its salts are is an anti-microbial effective against a wide-range of Gram-positive and Gram-negative bacteria.

While chlorhexidine may be used as an anti-microbial in the topical composition of the present invention, a salt of chlorhexidine is preferred due to its solubility properties. Preferred salts of chlorhexidine include chlorhexidine gluconate, chlorhexidine acetate and chlorhexidine hydrochloride. The most preferred anti-microbial for the topical composition of the present invention is chlorhexidine gluconate because it has excellent instantaneous bacterial effect that persists after use.

The amount of anti-microbial present in the topical composition should provide a balance between the desired antibacterial effect and unwanted adverse effects.

The anti-microbial is preferably present in an amount of about 3 g/L to about 10 g/L, more preferably about 4 g/L to about 7 g/L, most preferably about 5 g/L of the topical composition.

While a single anti-microbial may be used in the topical composition of the present invention, additional anti-microbials may be added to enhance the anti-microbial action of the topical composition. This may be particularly desirable in critical uses such as preparatory to invasive procedures.

Emollients

Emollients are typically added to topical compositions because they act to increase the moisture content of the stratum corneum.

Emollients are generally separated into two broad classes based on their function. The first class of emollients function by forming an occlusive barrier to prevent water evaporation from the stratum corneum. The second class of emollients penetrate into the stratum corneum and physically bind water to prevent evaporation. The first class of emollients is subdivided into compounds that are waxes at room temperature and compounds that are liquid oils. The second class of emollients includes those that are water-soluble and are often referred to as humectants.

The emollient may be selected from the following non-limiting list of general emollients, occlusive emollients and humectants.

Examples of general emollients includes short chain alkyl or aryl esters (C₁-C₆) of long straight or branched chain alkyl or alkenyl alcohols or acids (C₈-C₃₂) and their polyethoxylated derivatives; short chain alkyl or aryl esters (C₁-C₆) or C₄-C₁₂ diacids or diols optionally substituted in available positions by —OH; alkyl or aryl C₁-C₁₀ esters of glycerol, pentaerythritol, ethylene glycol, propylene glycol, as well as polyethoxylated derivatives of these and polyethylene glycol; C₁₂-C₂₂ alkyl esters or ethers of polypropylene glycol; C₁₂-C₂₂ alkyl esters or ethers of polypropylene glycol/polyethylene glycol copolymer; and polyether polysiloxane copolymers.

Examples of occulsive emollients include cyclic and linear dimethicones, polydialkysiloxanes, polyaryl/alkylsiloxanes, long chain (C₈-C₃₆) alkyl and alkenyl esters of long straight or branched chain alkyl or alkenyl alcohols or acids; long chain (C₈-C₃₆) alkyl and alkenyl amides of long straight or branched chain alkanes and alkenes such as squalene, squalane and mineral oil; jojoba oil polysiloxane polyalkylene copolymers, dialkoxy dimethyl polysiloxanes, short chain alkyl or aryl esters (C₁-C₆) of C₁₂-C₂₂ diacids or diols optionally substituted in available positions by —OH, such as diisostearyl dimer dilinoleate; lanolin and lanolin derivatives, and beeswax and its derivatives.

Examples of humectant type emollients include glycerol, propylene glycol, dipropylene glycol, polypropylene glycol, polyethylene glycol, sorbitol, pantothenol, gluconic acid salts and the like.

Humectant type emollients are preferred for the topical composition of the present invention. The most preferred humectant is polyethylene glycol. Polyethylene glycol is pleasant to use, readily available and economical.

The concentration of the emollient in the topical composition is preferably about 1.0% to about 2.0%, more preferably about 1.5% to about 1.8%, most preferably about 1.5%.

The inventors have determined that having a 1.5% concentration of polyethylene glycol in the topical composition of the present invention provides adequate moisturizing while not causing irritation and/or allergic reactions. There are also no unwanted abnormal feelings after application such as sliminess or stickiness.

Additional Ingredients

The topical composition may optionally comprise ingredients such as water, oils, salts, fragrances, perfumes, colorants, stabilizers, emulsifiers, propellants, additives, preservatives or preserving agents, anti-oxidants, surfactants, thickeners and other excipients normally used in topical compositions. If additional ingredients are included in the topical composition, ingredients that are known to cause skin irritation and/or sensitization reactions should be avoided.

Formulations

The topical composition may be formulated into the form of an aerosol, balm, cream, emolument, foam, gel, liniment, lotion, ointment, salve, solution, spray, suspension, unguent or the like.

Application

The present invention is a topical composition, which is useful as a broad-spectrum antiseptic and/or disinfectant for use in many environments including hospitals and clinics, veterinarian, industrial, food industry, livestock and home environments. The topical composition may be used, for example, as a skin disinfectant.

The topical compositions of the present invention are highly efficacious in preventing the transmission of microorganisms within hospitals.

The term “microorganism” includes any microscopic organism or taxonomically related macroscopic organism within the categories algae, bacteria, fungi, protozoa, viruses and subviral agents or the like. The microorganism is preferably a multi-resistant organism such as, for example, methicillin-resistant staphylococcus aureus.

In addition, preferred topical compositions of this invention maintain moisture after both single and multiple applications without allergic reaction or unwanted side effects such as abnormal feelings of sliminess or stickiness.

Topical compositions of the present invention are suitable for frequent and repeated usage during routine and specialized health-care delivery in environments such as hospital or clinic. In hospitals and clinics, these topical compositions may be used before and after each patient contact or preparatory to invasive procedures.

If topical compositions of the present invention are formulated into a hand lotion, only a small amount, about 1 ml to about 3 ml, of the hand lotion would be needed to be effective disinfectant and/or antiseptic.

Topical compositions of the present invention have advantages over known topical compositions in that they may be applied frequently with minimal adverse effects, dry rapidly, provide both instantaneous and prolonged anti-microbial activity, and have a low toxicity profile.

The term “topical composition” as used herein generally refers to a composition that is applied externally to any part of the body excluding mucous membranes such as the eyes, mouth, and so on. The topical composition may, therefore, be applied directly to any part of the body excluding mucous membranes such as the eyes, mouth, and so on. However, topical compositions of the present invention may also be incorporated into sponges, swabs, pads and/or wipes, which are then used to apply the topical solution to any part of the body excluding mucous membranes such as the eyes, mouth, and so on. The topical compositions may also be incorporated into cosmetic products.

Stability

The topical composition of the invention conserves its activity for, at least, two years from its production. However, it is preferable to use it at least within 12 months of being prepared. Even so, incorporating appropriate preserving agents can extend the period to sustain activity.

### EXAMPLES

The invention will now be further described with reference to the following Examples. These Examples are not intended to limit the invention in any way.

The topical composition of the present invention may be prepared by a variety of techniques.

One method of preparing the topical composition of the present invention involves mixing the components of the topical composition, in suitable quantities, with stirring, until complete homogenization is achieved.

The following examples are topical compositions of the present invention that were prepared according to the above method.

### Example 1

| | |
|---|---|
| isopropyl alcohol | 70% v/v |
| chlorhexidine gluconate | 5 g/L |
| polyethylene glycol | 1.5% concen. |
| water | balance |

The topical composition of Example 1 was formulated into the form of a lotion, in particular a hand lotion.

The hand lotion was shown to be an effective disinfectant and/or antiseptic. It was also gentle on the skin, dried rapidly and provided both instantaneous and prolonged anti-microbial activity. The hand lotion showed no unwanted side effects such as abnormal feelings of sliminess or stickiness.

The hand lotion of Example 1 was also trialed as a disinfectant hand lotion in an intervention/study ward of the Austin & Repatriation Medical Centre, Victoria, Australia.

The trial was conducted over a period of ten or so months.

The staff of the intervention/study ward were instructed to use about 1 ml to about 3 ml of the disinfectant hand lotion before and after each patient contact.

The results of the trial showed that there was an approximately 21% to 25% reduction in clinical infections with methicillin-resistant staphylococcus aureus (“MRSA”).

Since the disinfectant hand lotion was easy to use, there was also a 25% to 100% improvement in hand hygiene compliance. Hand hygiene compliance refers to the rates of hand washing, which in the present case would refer to the rates of use of the topical composition as a disinfectant hand lotion.

Furthermore, there were no allergic reactions identified through repeated use of the hand lotion during the trial.

Accordingly, this trial showed that the topical composition of the present invention would be very effective as a disinfectant hand lotion in hospitals, clinics, surgical and medical centers and the like.

### Example 2

| | |
|---|---|
| isopropyl alcohol | 70% v/v |
| chlorhexidine gluconate | 5 g/L |
| polyethylene glycol | 2.0% concen. |
| water | balance |

The topical composition of Example 2 was formulated into the form of a lotion, in particular a hand lotion.

The hand lotion was shown to be an effective disinfectant and/or antiseptic. It was also gentle on the skin, dried rapidly and provided both instantaneous and prolonged anti-microbial activity.

The hand lotion was found, however, to have a slight unwanted after effect.

Specifically, after repeated use of the hand lotion, there was a feeling of stickiness.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The invention claimed is:

1. A topical composition comprising:
at least one C₁ to C₄ alcohol;
at least one anti-microbial; and
at least one emollient.

2. A topical composition according to claim 1, wherein the C₁ to C₄ alcohol is a straight or branched chain alcohol.

3. A topical composition according to claim 1, wherein the alcohol is selected from ethanol, n-propanol, isopropanol or a combination thereof.

4. A topical composition according to claim 3, wherein the alcohol is isopropanol.

5. A topical composition according to claim 1, wherein the alcohol concentration is about 60% v/v to about 90% v/v of the total concentration of the topical composition.

6. A topical composition according to claim 5, wherein the alcohol concentration is about 65% v/v to about 75% v/v of the total concentration of the topical composition.

7. A topical composition according to claim 6, wherein the alcohol concentration of the topical composition is about 70% v/v of the total concentration of the topical composition.

8. A topical composition according to claim 1, wherein the anti-microbial is selected from chlorhexidine and its salts.

9. A topical composition according to claim 1, wherein the anti-microbial is selected from chlorhexidine gluconate, chlorhexidine acetate and chlorhexidine hydrochloride.

10. A topical composition according to claim 9, wherein the anti-microbial is chlorhexidine gluconate.

11. A topical composition according to claim 1, wherein additional anti-microbials are added to the topical composition to enhance its anti-microbial action.

12. A topical composition according to claim 1, wherein the anti-microbial is present in an amount of about 3 g/L to about 10 g/L of the topical composition.

13. A topical composition according to claim 12, wherein the anti-microbial is present in an amount of about 4 g/L to about 7 g/L of the topical composition.

14. A topical composition according to claim 13, wherein the anti-microbial is present in an amount of about 5 g/L of the topical composition.

15. A topical composition according to claim 1, wherein the emollient is selected from general emollients, occlusive emollients and humectants.

16. A topical composition according to claim 15, wherein the emollient is a general emollient selected from short chain alkyl or aryl esters (C₁-C₆) of long straight or branched chain alkyl or alkenyl alcohols or acids (C₈-C₃₂) and their polyethoxylated derivatives; short chain alkyl or aryl esters (C₁-C₆) or C₄-C₁₂ diacids or diols optionally substituted in available positions by —OH; alkyl or aryl C₁-C₁₀ esters of glycerol, pentaerythritol, ethylene glycol, propylene glycol, as well as polyethoxylated derivatives of these and polyethylene glycol; C₁₂-C₂₂ alkyl esters or ethers of polypropylene glycol; C₁₂-C₂₂ alkyl esters or ethers of polypropylene glycol/polyethylene glycol copolymer; and polyether polysiloxane copolymers.

17. A topical composition according to claim 15, wherein the emollient is an occlusive emollient selected from cyclic and linear dimethicones, polydialkysiloxanes, polyaryl/alkylsiloxanes, long chain (C₈-C₃₆) alkyl and alkenyl esters of long straight or branched chain alkyl or alkenyl alcohols or acids; long chain (C₈-C₃₆) alkyl and alkenyl amides of long straight or branched chain alkanes and alkenes such as squalene, squalane and mineral oil; jojoba oil polysiloxane polyalkylene copolymers, dialkoxy dimethyl polysiloxanes, short chain alkyl or aryl esters (C₁-C₆) of C₁₂-C₂₂ diacids or diols optionally substituted in available positions by —OH, such as diisostearyl dimer dilinoleate; lanolin and lanolin derivatives, and beeswax and its derivatives.

18. A topical composition according to claim 15, wherein the emollient is a humectant selected from glycerol, propylene glycol, dipropylene glycol, polypropylene glycol, polyethylene glycol, sorbitol, pantothenol and gluconic acid salts.

19. A topical composition according to claim 18, wherein the emollient is polyethylene glycol.

20. A topical composition according to claim 1, wherein the concentration of the emollient in the topical composition is about 1.0% to about 2.0%.

21. A topical composition according to claim 20, wherein the concentration of the emollient in the topical composition is about 1.5% to about 1.8%.

22. A topical composition according to claim 21, wherein the concentration of the emollient in the topical composition is about 1.5%.

23. A topical composition according to claim 1, which further comprises water, oils, salts, fragrances, perfumes, colorants, stabilizers, emulsifiers, propellants, additives, preservatives or preserving agents, anti-oxidants, surfactants, thickeners and other excipients normally used in topical compositions.

24. A topical composition according to claim 1, wherein the topical composition is formulated into the form of an aerosol, balm, cream, emolument, foam, gel, liniment, lotion, ointment, salve, solution, spray, suspension, unguent or the like.

25. A topical composition according to claim 1, wherein the topical composition is incorporated into sponges, swabs, pads and/or wipes.

26. A topical composition according to claim 1, wherein the topical composition is incorporated into cosmetic products.

27. A topical composition according to claim 1, wherein the topical composition is used on any part of the body excluding mucous membranes.

28. A method of reducing and/or preventing the transmission of a microorganism by applying an effective amount of a topical composition comprising at least one C₁ to C₄ alcohol, at least one anti-microbial, and at least one emollient to any part of the body excluding mucous membranes.

29. The method according to claim 28, wherein the microorganism is a multi-resistant organism.

30. The method according to claim 29, wherein the multi-resistant organism is methicillin-resistant staphylococcus aureus.

31. The method according to claim 28, wherein the topical composition is applied before and after routine or specialized health-care delivery or preparatory to invasive procedures.

32. The method according to claim 28, wherein an effective amount is about 1 ml to about 3 ml.

33. The method of reducing and/or preventing the transmission of a microorganism comprising applying a topical composition comprising at least one C₁ to C₄ alcohol, at least one anti-microbial, and at least one emollient.

34. The method of reducing and/or preventing the transmission of a microorganism according to claim 33, wherein the microorganism is a multi-resistant organism.

35. The method of reducing and/or preventing the transmission of a microorganism according to claim 34, wherein the multi-resistant organism is methicillin-resistant staphylococcus aureus.

36. The method of reducing and/or preventing the transmission of a microorganism according to claim 33, wherein the topical composition is used before and after routine or specialized health-care delivery or preparatory to invasive procedures.

37. The method of reducing and/or preventing the transmission of a microorganism according to claim 33, wherein about 1 ml to about 3 ml of the topical composition is used.

38. The method of reducing and/or preventing the transmission of a microorganism comprising at least one C₁ to C₄ alcohol, at least one anti-microbial, and at least one emollient and topically applying the disinfectant and/or antiseptic.

39. The method of reducing and/or preventing the transmission of a microorganism according to claim 38, wherein the microorganism is a multi-resistant organism.

40. The method of reducing and/or preventing the transmission of a microorganism according to claim 39, wherein the multi-resistant organism is methicillin-resistant staphylococcus aureus.

41. A disinfectant and/or antiseptic comprising a topical composition comprising:
at least one C₁ to C₄ alcohol;
at least one anti-microbial; and
at least one emollient.

42. A method of preparing a topical composition comprising mixing at least one C₁ to C₄ alcohol, at least one anti-microbial, and at least one emollient, with stirring, until complete homogenization is achieved.
